(19) United States
(12) Patent Application Publication
(10) Pub. No.: US 2004/0122433 A1
(43) Pub. Date: Jun. 24, 2004

(54) Endoscopic rongeur-type surgical instrument

(76) Inventors: Thierry Loubens, Saint Didier Au Mont D'Or (FR); Christophe Garin, Lyon (FR); Lionel Riou, Lyon (FR)

Correspondence Address: YOUNG & THOMPSON, 745 SOUTH 23RD STREET 2ND FLOOR, ARLINGTON, VA, 22202

(21) Appl. No.: 10/467,314

(22) PCT Filed: Feb. 1, 2002
(86) PCT No.: PCT/FR02/00388

(30) Foreign Application Priority Data
Feb. 6, 2001 (FR) 01/01577

(51) Int. Cl. A61B 17/16

(52) U.S. Cl. 606/83

(57) ABSTRACT

The invention relates to a rongeur-type surgical instrument comprising a fixed handle (2) which receives an elastically-loaded movable handle (4) around an axis of rotation (5), a fixed clamping jaw (3), one end of which is solidly connected to a cutting jaw, (25) and a movable clamping jaw (6). The movable clamping jaw moves in translation on the fixed clamping jaw (3) when the movable handle (4) pivots around the axis of rotation (5) in such a way that the sharp edges (45) of the free end thereof co-operate with the sharp edges (27) of the fixed clamping jaw (3). Said movable clamping jaw (6) comprises an internal channel (44) which stores the fragments of cut bone tissue (56), while the fixed clamping jaw (3) comprises transfer means (30) which co-operate with the movable clamping jaw (6) in order to enable the fragments of cut bone tissue (56) to penetrate a space (41) in the movable clamping jaw (6) which is disposed in the extension of the channel (44).

The present invention relates to a surgical instrument more commonly known as KERRISSON forceps, intended for example, but without obligation, for endoscopic operations via the anterior route.

Patent DE 29718969 for example discloses a surgical instrument of the punch type comprising a main body forming a fixed jaw, a moving jaw moving on the fixed one via an elastically loaded handle.

The moving handle pivots about an axis of rotation on the one hand and has elastic return means connecting said moving handle to a fixed handle of the main body, on the other.

It may be noted that the elastic return means limit the travel of the moving handle about its axis of rotation. The travel of the moving handle is limited forward by stops created by the main body and the removable stop of the head of the axle, and at the rear by the moving jaw, which butts against the nose of the fixed jaw.

Extending the forward travel of the moving handle, when the removable stop of the axle is deactivated, allows the drive means for driving the moving jaw to be positioned in a given angular position in order to allow said jaw either to be set in place or to be withdrawn.

Furthermore, this surgical instrument of the punch type is not intended for endoscopic operations via the anterior route.

This surgical instrument of the rongeur type according to the present invention comprises a fixed handle receiving, about an axis of rotation, an elastically loaded moving handle, a fixed jaw secured at one of its ends to a nose with a cutting profile, a moving jaw guided in translation on the fixed jaw as the moving handle pivots about the axis of rotation so that its free end with cutting edges collaborates with those of the fixed jaw, said moving jaw comprising an internal passage forming a store for the cut pieces of bone tissue, while the fixed jaw comprises transfer means which collaborate with the moving jaw to allow the cut pieces of bone tissue to enter a space belonging to the moving jaw and situated in the continuation of the passage.

The surgical instrument of the rongeur type according to the present invention comprises a moving jaw which consists of a cylindrical tube comprising an open internal bore, of a continuation and of a ring which is formed of a first part with a knurled surface and of a second part provided with oblong housings in which drive spindles are fixed.

The surgical instrument of the rongeur type according to the present invention comprises a fixed jaw which consists of a cylindrical tube comprising an internal bore of a continuation in the form of an elongate mounting plate having a U-shaped solid cross section and of a cylindrical shoulder secured to a stop.

The surgical instrument of the rongeur type according to the present invention comprises transfer means which consist of a shoulder secured to the fixed jaw and which enters a space of the moving jaw in the operating position.

The surgical instrument of the rongeur type according to the present invention comprises a shoulder which comprises an elongate part arranged inside the bore and above the internal wall of the cylindrical tube of the fixed jaw so as to delimit a slot of curved profile in which the wall of the cylindrical tube of the moving jaw slides in the operating position.

The surgical instrument of the rongeur type according to the present invention comprises a continuation which comprises, over its entire length, an internal passage in the shape of an inverted U with an open base.

The surgical instrument of the rongeur type according to the present invention comprises a continuation provided at its free end with inclined and opposed edges which are beveled.

The surgical instrument of the rongeur type according to the present invention comprises a passage which comprises, near the inclined edges, opposed ribs separated by a slit so as to constitute a slideway.

The surgical instrument of the rongeur type according to the present invention comprises a ring which is provided with oblong housings which are made parallel to the longitudinal axis of the moving jaw, while drive spindles are fixed in each housing and directed in a direction perpendicular to the longitudinal axis so as to collaborate with the moving handle for driving the translation of the moving jaw.

The surgical instrument of the rongeur type according to the present invention comprises oblong housings which are uniformly distributed around the periphery of the ring.

The surgical instrument of the rongeur type according to the present invention comprises a ring which is provided with just one oblong housing and just one drive spindle while the tube is free to rotate about the ring, allowing the moving jaw to automatically position itself according to the angular indexing of the fixed jaw relative to the fixed handle.

The surgical instrument of the rongeur type according to the present invention comprises a continuation which comprises an upper edge which has, at the shoulder and inside the tube, lateral stops delimiting, on the central axis of the fixed jaw, a groove for the longitudinal guidance of the moving jaw in the operating position.

The surgical instrument of the rongeur type according to the present invention comprises a continuation which comprises, at its free end, a nose which in its thickness has a hollowing delimiting opposed and beveled edges.

The surgical instrument of the rongeur type according to the present invention comprises a continuation which comprises an upper edge provided, near the nose, with a T-shaped tenon allowing longitudinal guidance of the moving jaw in the operating position.

The surgical instrument of the rongeur type according to the present invention comprises a wall of the cylindrical tube of the fixed jaw which is pierced with a hole which communicates with the slot of curved profile.

The surgical instrument of the rongeur type according to the present invention comprises a tube which comprises, at the shoulder, a nut provided in its internal part with a groove which collaborates with the stop to allow the fixed jaw to be fixed indexed in a given angular position on the fixed handle.

The surgical instrument of the rongeur type according to the present invention comprises a fixed handle which comprises fixing means that allow the fixed jaw to be arranged in different angular positions about its longitudinal axis.

The surgical instrument of the rongeur type according to the present invention comprises fixing means which consist of a cylindrical sleeve tube comprising at one of its ends a threaded collar delimiting a shoulder in which cut-outs are made, at least one of which collaborates, according to the determined position, with a stop secured to the fixed jaw, while the threaded collar collaborates with a tightening nut for immobilizing the fixed jaw on the fixed handle.

The surgical instrument of the rongeur type according to the present invention comprises a cylindrical sleeve tube which comprises an internal bore open at both ends and communicating with a slit of conical profile formed in the thickness of the upper part of the fixed handle.

The surgical instrument of the rongeur type according to the present invention comprises a slit which is designed to accommodate a mounting plate of the moving handle in order to open into the internal bore of the sleeve tube so as to be able to collaborate with the spindle or one of the spindles of the moving jaw.

The surgical instrument of the rongeur type according to the present invention comprises a mounting plate of the moving handle comprising an oblong slit intended to accommodate one of the spindles of the moving jaw in the operating position.

The description which will follow with reference to the attached drawings, given by way of nonlimiting examples, will allow for a better understanding of the invention, of the features it has and of the advantages it is likely to afford:

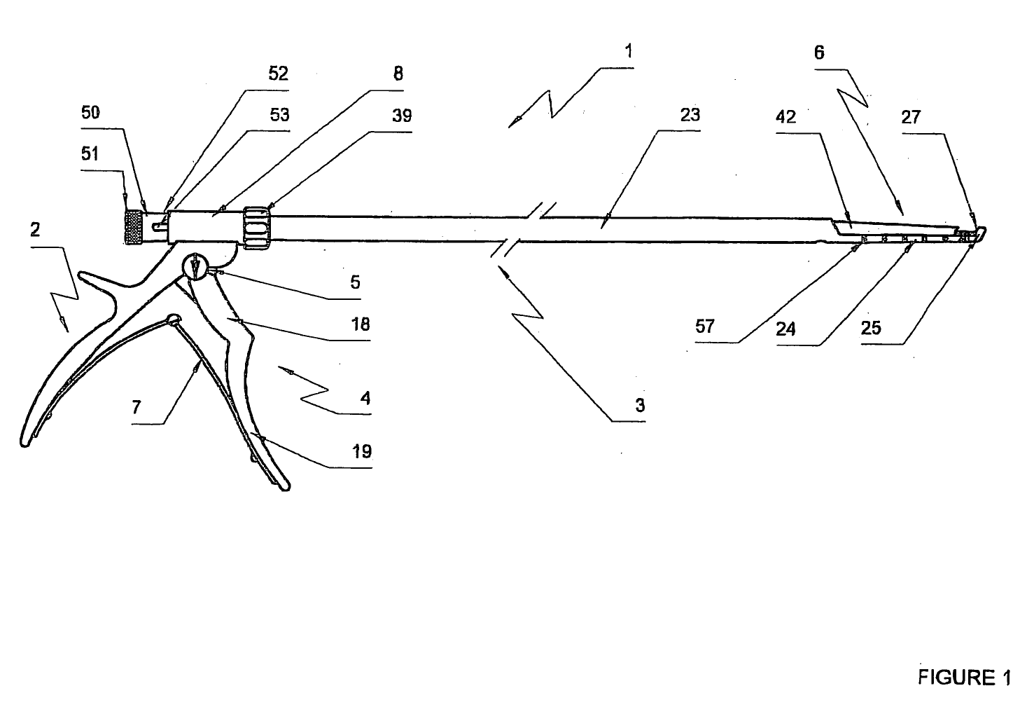

FIG. 1 is a view illustrating the surgical instrument according to the present invention.

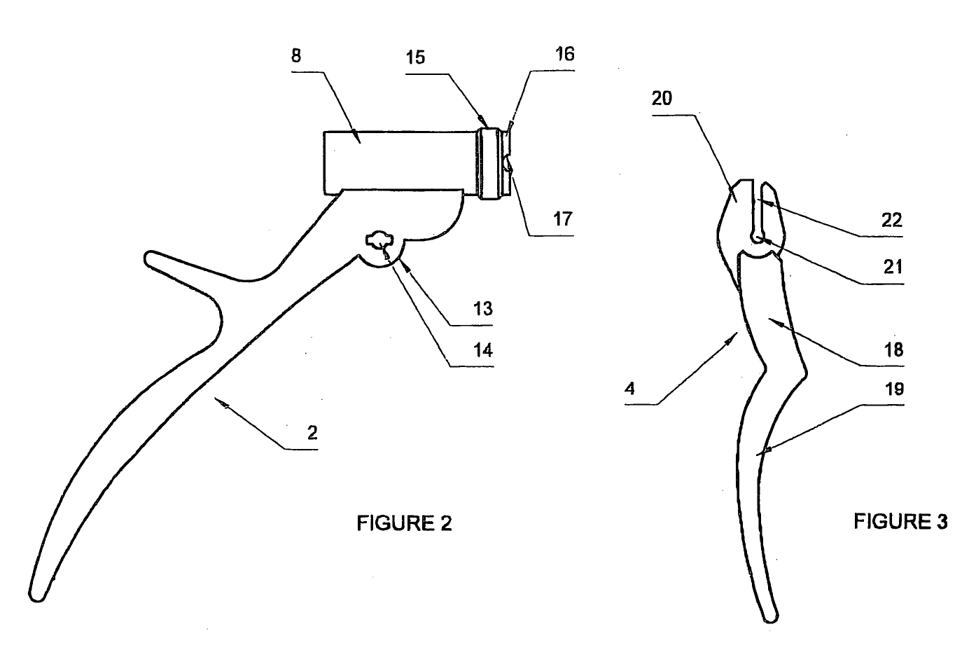

FIG. 2 is a view showing the fixed handle and the fixing means for the fixed jaw of the surgical instrument according to the present invention.

FIG. 3 is a view depicting the moving handle of the surgical instrument according to the present invention.

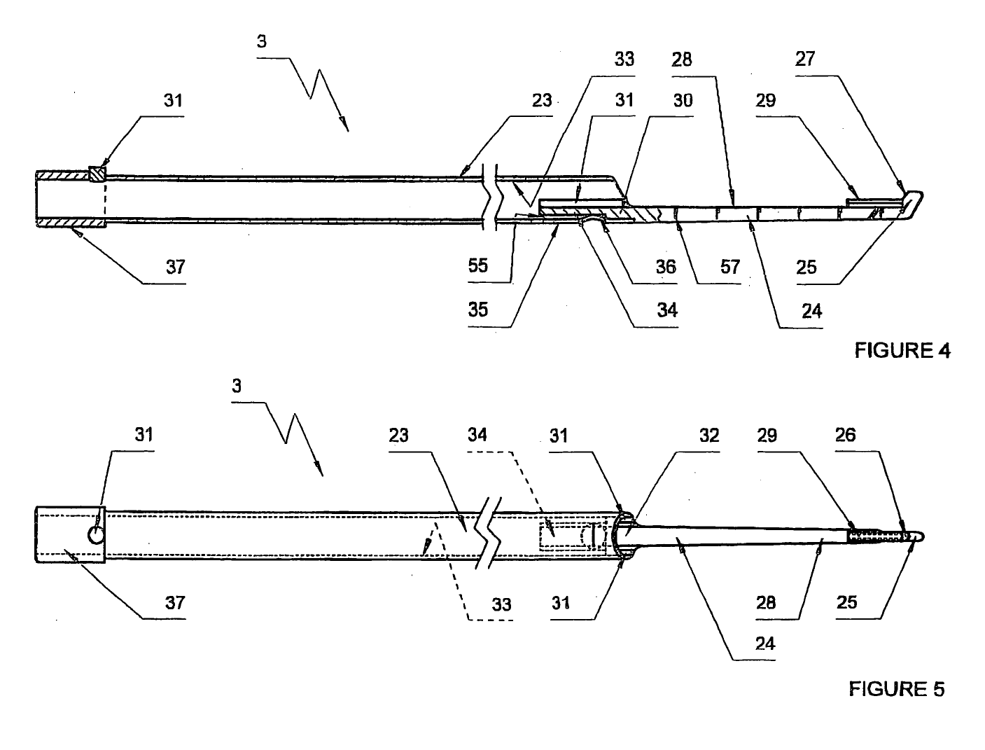

FIGS. 4 and 5 are views illustrating the fixed jaw of the surgical instrument according to the present invention.

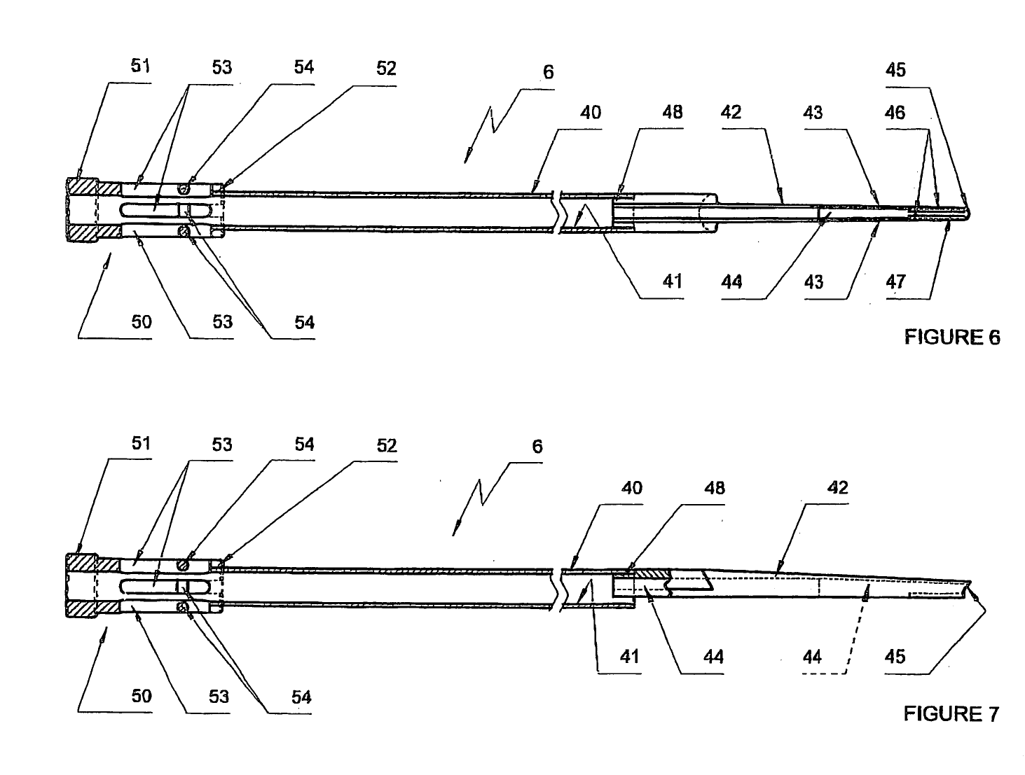

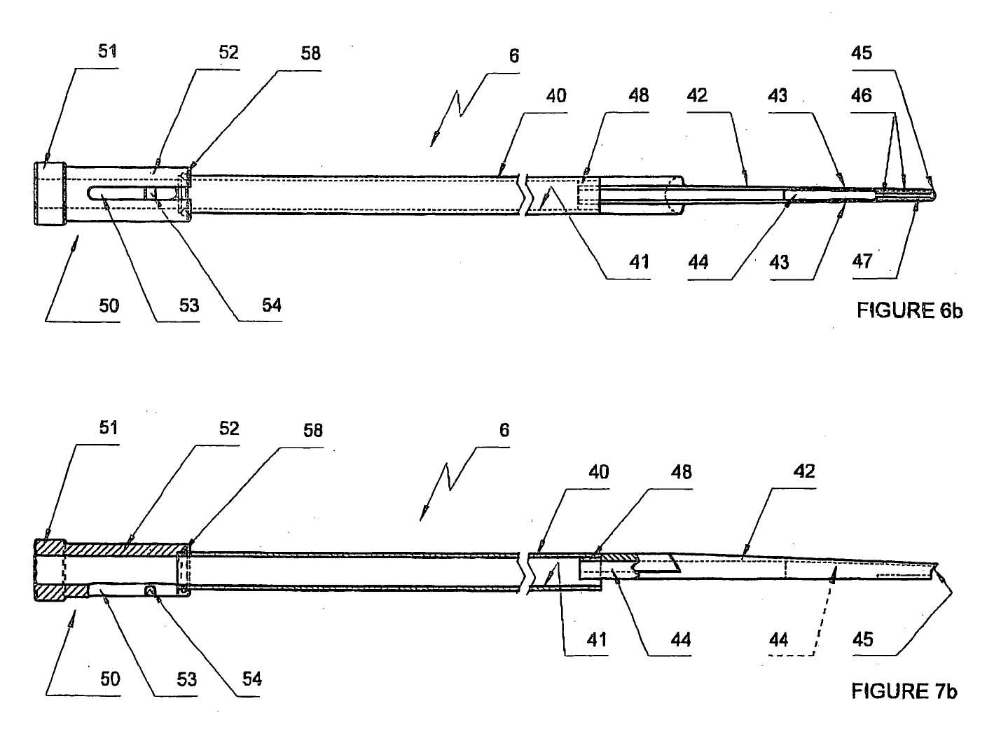

FIGS. 6 and 7 are views showing the moving jaw of the surgical instrument according to the present invention.

FIGS. 6b and 7b are views depicting an alternative form of the means for driving the moving jaw of the surgical instrument according to the present invention.

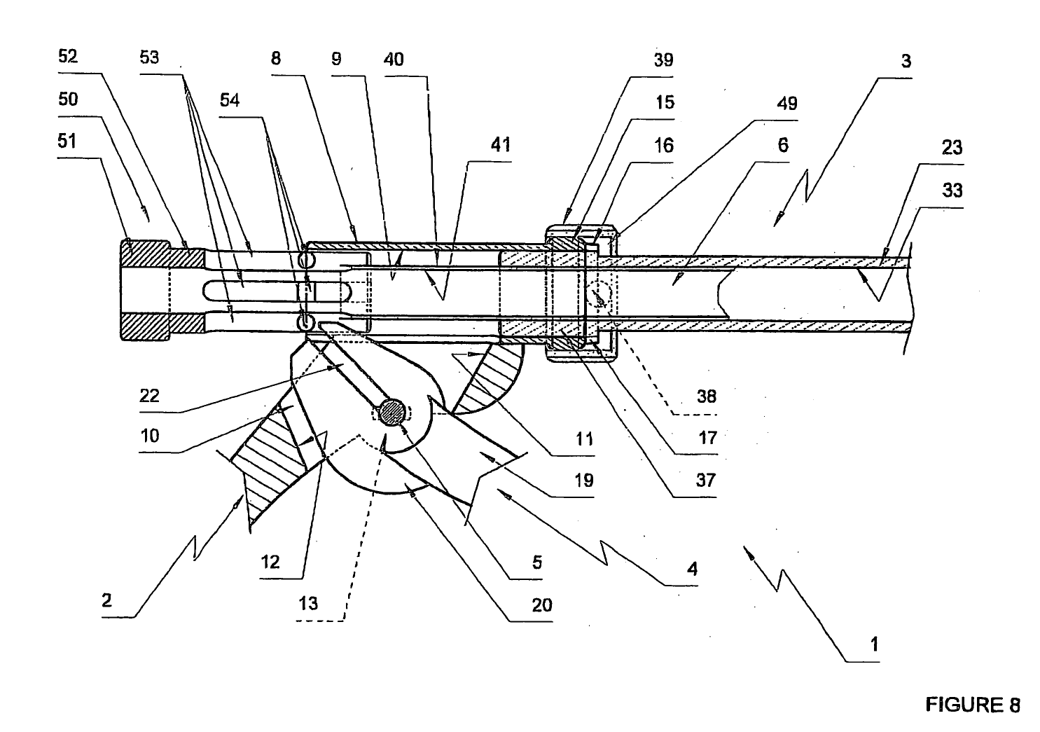

FIG. 8 is a view depicting the collaboration of the fixed and moving jaws with the fixed and moving handles of the surgical instrument according to the present invention.

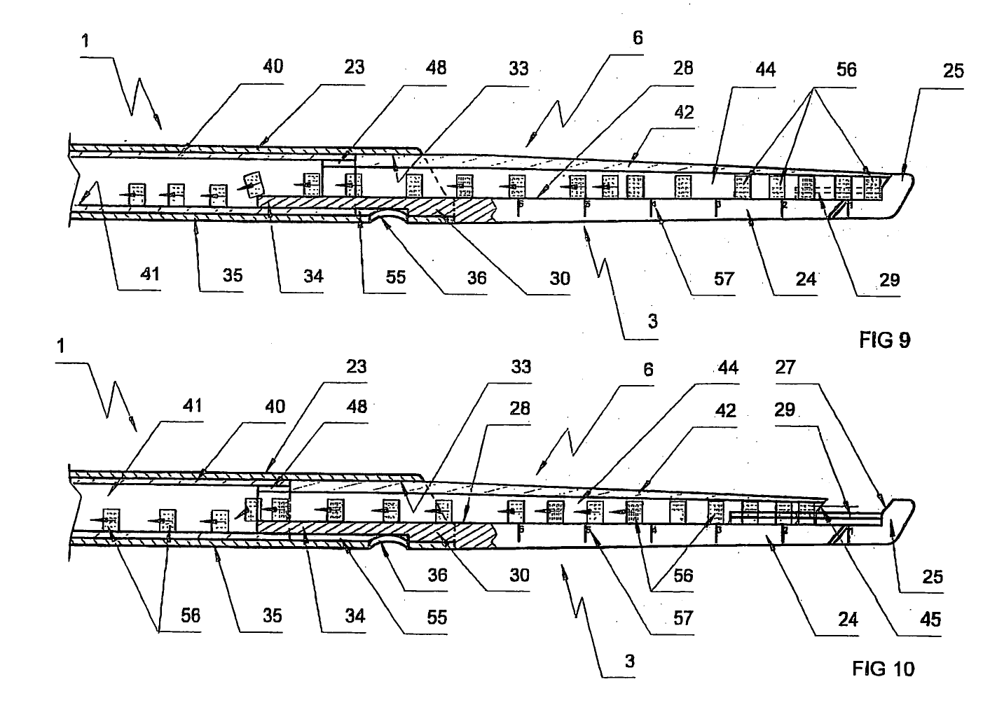

FIGS. 9 and 10 are schematic views illustrating the movement of the moving jaw inside the fixed jaw and the transfer of the pieces of bone tissue.

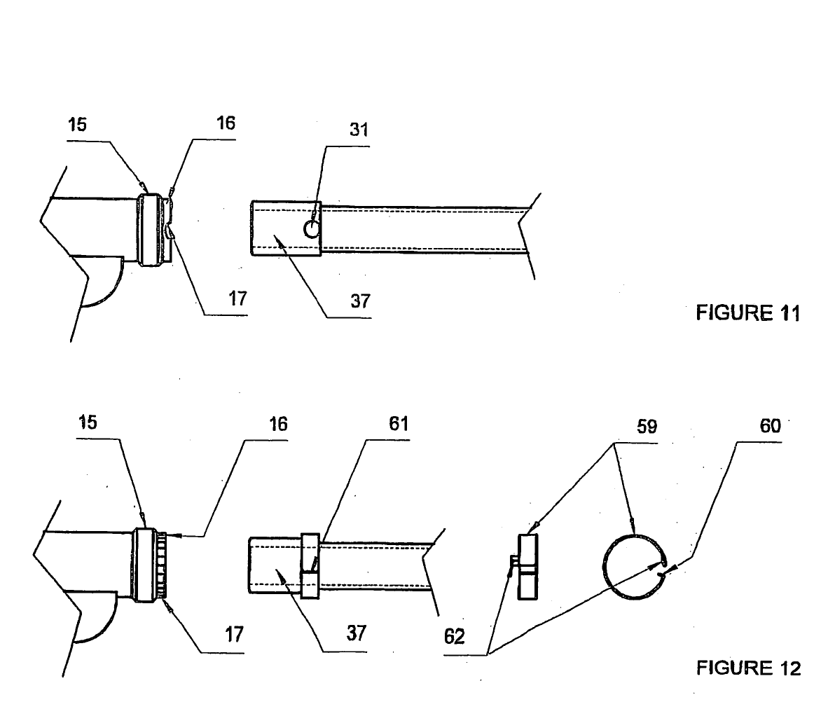

FIGS. 11 and 12 are views showing an alternative form of the means of fixing the fixed jaw on the fixed handle of the surgical instrument according to the present invention.

FIG. 1 shows a surgical instrument 1 of the “KERRISSON” forceps or rongeur type comprising a main body formed of a fixed handle 2 on which a fixed jaw 3 is fixed in a roughly horizontal plane.

The surgical instrument 1 comprises a moving handle 4 which pivots about an axis of rotation 5 passing through the fixed handle 2 for the longitudinal movement of a moving jaw 6.

The moving handle 4 is connected to the fixed handle 2 by a leaf-shaped spring 7 to place the moving handle 4 in a single starting position after each pivoting action.

The fixed jaw 3 of the surgical instrument 1 collaborates with the moving jaw 6 which slides in the longitudinal direction guided back and forth on said fixed jaw 3 when force is applied to the moving handle 4.

FIG. 2 shows the fixed handle 2 of the surgical instrument 1 which is secured in its upper part and in a horizontal direction to fixing means which consist of a hollow cylindrical sleeve tube 8.

The cylindrical sleeve tube 8 comprises an internal bore 9 open at both ends and communicating with a slit 10 of conical profile formed in the thickness of the upper part of the fixed handle.

The slit 10 is delimited by opposed edges 11, 12 so that the most open side of said slit faces toward the cylindrical sleeve tube 8.

The fixed handle 2 comprises, on each side of the slit 10, a lug 13 in the shape of a portion of a circle pierced with a bore 14 in the shape of a keyhole intended to accommodate the axis of rotation 5 for guiding the rotation of the moving handle 4.

The maximum travel of the moving handle 4 about the axis of rotation 5 inside the slit 10 of the fixed handle 2 is delimited by the opposed edges 11, 12 as already described in patent applications in the name of the applicant.

The cylindrical sleeve tube 8 is given a length greater than that of the upper part of the fixed handle 2 so that each free end of said sleeve tube is easy to access.

The cylindrical sleeve tube 8 comprises at one of its ends a threaded collar 15 delimiting a shoulder 16 in which cut-outs 17 are made, uniformly distributed around the peripheral perimeter of said sleeve tube.

FIG. 3 illustrates the moving handle 4 of the surgical instrument 1 which has a cranked profile consisting of a first branch 18, of short length, extended by a second branch 19, of longer length.

The first branch 18 is extended at the opposite end to the second branch 19 by a mounting plate 20 forming a fork pierced with a bore 21 which communicates with an open oblong slit 22.

The bore 21 has a diameter slightly greater than that of the oblong slit 22.

The bore-21 is designed to accommodate the axis of rotation 5 so that the moving handle 4 can pivot about this axis when mounted in the slit 10 of the fixed handle 2 of the surgical instrument 1.

It will be noted that the free end of the oblong slit 22 formed in the mounting plate 20 opens into the bore 9 of the sleeve tube 8 of the fixed handle 2 to collaborate with the moving jaw 6.

FIGS. 4, 5 and 11 depict the fixed jaw 3 of the surgical instrument 1, which jaw consists of a cylindrical tube 23 secured at one of its ends to a continuation 24 in the form of an elongate mounting plate that has a U-shaped solid cross section.

The continuation 24 comprises, at its free end, a nose 25 having, in its thickness, a hollowing 26 delimiting opposed edges 27 which are inclined with respect to the upper edge 28 of said continuation.

The edges 27 of the nose 25 are machined to constitute cutting elements so as to cut bone fragments.

The continuations 24 comprise, near the nose 25, a T-shaped tenon 29 allowing longitudinal guidance of the moving jaw 6 along its entire travel.

The continuation 24 comprises on its two faces graduations 57 intended to guide the operator in the endoscopic operations.

The continuation 24 comprises, at the opposite end to its nose 25, a shoulder 30 which enters the lower part of the cylindrical tube 23 and part of the shoulder of which is welded into said tube to form a rigid and firm connection.

The upper edge 28 of the continuation 24 comprises, at the shoulder 30 and inside the tube 23, lateral stops 31 delimiting, on the central axis of the fixed jaw 3, a slot 32 for the longitudinal guidance of the moving jaw 6.

The upper edges of the lateral stops 31, parallel to the edge 28, delimit the opening of the internal bore 33 of the tube 23 at the extension 24 of the fixed jaw 3.

The shoulder 30 comprises an elongate part 34 which is arranged inside the bore 33 and above the internal wall 35 of the cylindrical tube 23 making it possible to delimit a slot 55 of curved profile which communicates with a hole 36 opening to the outside of said tube.

The hole 36 is to enable pressurized products to be injected to clean the bore 33 of the tube 23 forming the fixed jaw 3.

The tube 23 of the fixed jaw 3 comprises on its external perimeter and at the opposite side to the continuation 24, a cylindrical shoulder 37 secured to a stop 38.

The tube 23 comprises, at the shoulder 37, a nut 39 provided in its internal part with a shoulder 49 allowing the stop 38 to pass (FIG. 8).

The fixed jaw 3 is fixed on the fixed handle 2 when the stop 38 collaborates with the corresponding cut-out 17 and the nut 39 screwed onto the collar 15.

In a second version shown in FIG. 12, the tube 23 of the fixed jaw comprises on its external perimeter and at the opposite side to the continuation 24, a cylindrical double shoulder 37, part of which has a slit 61.

This slit corresponds with a stub 60 provided on an elastic ring 59 so as to immobilize this ring around the shoulder 37.

The tube 23 at the double shoulder 37 comprises a nut 39 provided in its internal part with a shoulder 49 allowing the passage of the elastic ring 59 mounted without impeding its radial expansion.

The fixed jaw 3 is fixed on the fixed handle 2 when an indexing finger 62 of the elastic ring 59 collaborates with one of the cut-outs 17 of the shoulder 16.

Thus it can be seen that the shoulder 16 of the fixed handle 2 has different cut-out profiles 17 which may be either radial or axial depending on the usage of the surgical instrument 1.

It can be seen that the fixed jaw 3, and more particularly its nose 25, can be arranged in different planes about its horizontal axis according to the surgeon's choice and the operation that is to be performed, depending on which angular position of cut-out 17 is adopted on the periphery of the sleeve tube 8 for securing this fixed jaw 3.

FIGS. 6 and 7 show the moving jaw 6 which consists of a cylindrical tube 40 comprising an open internal bore 41 into which there is welded, at one of the ends of said tube, a continuation 42 forming an elongate mounting plate the hollow cross section of which is in the form of an inverted U.

The outer edges 43 of the continuation along the entire length of the continuation 42 delimit an internal passage 44 in the shape of an inverted U, the base of which is open.

The continuation 42 at its free end comprises inclined and opposed edges 45 which are beveled, outward on the one hand with respect to the outer edge 43 and toward the inside of the passage 44 on the other.

The passage 44 comprises, near the inclined edges 45, opposed ribs 46 separated by a slit 47 so as to form a slideway collaborating with the T-shaped tenon 29 of the continuation 24 of the fixed jaw 3.

On the opposite side to the inclined edges 45, the continuation 42 has a shoulder 48 welded into the upper and internal part of the cylindrical tube 40 to form a rigid and firm connection.

The inverted U-shaped internal passage 44 opens into the internal bore 41 of the cylindrical tube 40 so that its open base faces toward the lower part of said tube.

It will be noted that the internal profile of the passage 44 increases from the inclined edges 45 to the end of the shoulder 48.

It will be noted that the internal passage 44 is designed to constitute a store to store the bone fragments 56 cut using the beveled edges 27 and 45 of each fixed 3 and moving 6 jaw (FIG. 9).

The cylindrical tube 40 is secured on the opposite side to the extension 42 to a cylindrical ring 50 the outside diameter of which exceeds that of said tube.

The ring 50 consists of a first part 51 with a knurled surface for holding the moving jaw 6 and of a second part 52 provided with oblong housings 53 in which drive spindles 54 are fixed.

The oblong housings 53 are uniformly distributed about the periphery of the ring 50 in angular directions which are the same as those intended for the cut-outs 17 made in the periphery of the sleeve tube 8 of the fixed handle 2.

The oblong cut-outs 53 are made parallel to the longitudinal axis of the moving jaw 6, while the drive spindles 54 fixed in each housing are directed in a direction perpendicular to the longitudinal axis.

Before the moving jaw 6 is set in place inside the fixed jaw 3, the moving handle 4 is mounted in the fixed handle 2 by means of the axis of rotation 5 so that its oblong slit 22 is directed in a direction toward the rear of the instrument (FIG. 8).

The moving jaw 6 is introduced into the sleeve tube 8 secured to the moving handle 2 and on which the nut 39 is mounted for fixing the fixed jaw 3.

The continuation 42 of the moving jaw 6 sits above that 24 of the fixed jaw 3 so that the ribs 46 and the slit 47 collaborate with the tenon 29 provided near the nose 25 (FIG. 10).

Also while the moving jaw 6 is being set in place, it can be seen that the outer edges 43 are guided in the groove 32 provided on the shoulder 30 of the continuation 24 of the fixed jaw 3.

The moving jaw 6 is automatically indexed into an angular position by the guide means 29, 46, 47; 32, 43 according to the position of the fixed jaw 3 with respect to the fixed handle 2 so that one of the drive spindles 54 of the ring 50 enters the slit 22 of the moving handle 4 (FIG. 8).

FIGS. 6b and 7b show an alternative form of the ring 50 of the moving jaw 6 which consists in forming just one oblong housing 53 and just one drive spindle 54.

Here, the tube of the moving jaw 6 is free to rotate about the ring 50 allowing the moving jaw to position itself automatically according to the angular indexing of the fixed jaw 3 relative to the fixed handle 2.

For example, the tube 40 is crimped onto the ring 50 after a running band 58 that allows the tube to rotate with respect to the ring 50 has been fitted.

Finally, a hard point will allow the oblong housing 53 to be brought back along the axis of the tunnel 44 to make it easier to introduce the moving jaw 6, the fixed jaw 3 being oriented on the fixed handle 2 beforehand.

The moving handle 4 has various angular operating positions determined by the lateral displacement of the axis of rotation 5 as described either in patent EP 1 075 220 or in patent PCT/FR99 01528 of which the applicant is proprietor.

FIGS. 9 and 10 show the longitudinal movements in operation of the moving jaw 6 with respect to the fixed jaw 3 of the surgical instrument 1 when the surgeon operates the moving handle 4.

FIG. 10 depicts the surgical instrument 1 in its wide open position, that is to say when the beveled edges 45 of the moving jaw 6 are the furthest from those 27 of the nose 25 of the fixed jaw 3.

In this wide open position, it can be seen that the lower part of the cylindrical tube 40 of the moving jaw 6 collaborates with the slot of curved profile 55 delimited by the internal wall 35 of the tube 23 and the elongate part 34 of the shoulder 30 of the continuation 24 of the fixed jaw 3.

Again in this wide open position, it will be noted that the tenon 29 for guiding the continuation 24 of the fixed jaw 3 still collaborates with the slit 47 delimited by the ribs 46 of the continuation 42 of the moving jaw 6.

FIG. 10 illustrates the surgical instrument 1 in its closed position, that is to say when the beveled edges 27 and 45 of each jaw 3 and 6 are in pressed-together contact.

In this position, the surgeon has just cut a piece of tissue 56 or bone which is collected inside the internal passage 44 of the continuation 42 of the moving jaw 6, before this jaw returns through elastic return to the open position described and shown in FIG. 9.

Thus, the surgeon, by repeated movements on the moving handle 4, can move the moving jaw 6 with respect to the fixed jaw 3 between a wide open position and a closed position allowing him to cut the bone tissue 56.

These repeated actions allow the uniform and successive cutting of bone tissue 56 causing the internal passage 44 that forms a store for the storage of cut pieces gradually to become filled.

It can be noted that the cut pieces 56 are held laterally by the internal walls of the passage 44 of the continuation 42 of the moving jaw 6 and by the upper edge 28 of the continuation 24 of the fixed jaw 3.

As successive cuts are made by the surgeon, the passage 44 gradually fills and so it is essential for this passage to have sufficient capacity and also for the cut pieces 58 not to prevent the sliding of the moving jaw 6 with respect to the fixed jaw 3.

It can be seen that the cut pieces 56 contained in the passage 44 are pushed into this passage each time the instrument 1 is closed, by the next piece cut by the beveled edges 27 and 45.

These successive cutting operations will gradually convey the first cut pieces into the cylindrical tube 40 of the moving jaw, where the internal space delimited by the bore 41 is greater than the space in the passage 44.

Collecting the cut pieces 56 in the internal bore 41 makes it possible never to impede the sliding of the moving jaw 6 on the fixed jaw 3 because of the difference in size between said bore and the passage 44.

What actually happens is that the cut pieces 56 move smoothly along the passage 44 and along the upper edge 28 of the fixed jaw 3 and then become lodged in the bore 41 of the moving jaw 6 (FIG. 9).

Thus, the internal bore 41 of the tube 44 of the moving jaw 6 constitutes a large-capacity second store allowing the surgeon no longer to have to remove the surgical instrument 1 from the operating site with each cutting stroke.

It must also be understood that the foregoing description has been given merely by way of example and that it does not in any way restrict the field of the invention, a departure from the scope of which would not be made if the embodiment details described were replaced by any other equivalent.

1. A surgical instrument of the rongeur type comprising a fixed handle (2) receiving, about an axis of rotation (5), an elastically loaded moving handle (4), a fixed jaw (3) secured at one of its ends to a nose (25) with a cutting profile and a moving jaw (6) guided in translation on the fixed jaw (3) as the moving handle (4) pivots about the axis of rotation (5) so that its free end with cutting edges (45) collaborate with those (27) of the fixed jaw (3), characterized in that the moving jaw (6) comprises an internal passage (44) forming a store for the cut pieces of bone tissue (56), while the fixed jaw (3) comprises transfer means (30) which collaborate with the moving jaw (6) to allow the cut pieces of bone tissue (56) to enter a space (41) belonging to the moving jaw (6) and situated in the continuation of the passage (44).

2. The surgical instrument as claimed in claim 1, characterized in that the moving jaw (6) consists of a cylindrical tube (40) comprising an open internal bore (41), of a continuation (42) and of a ring (50) which is formed of a first part (51) with a knurled surface and of a second part (52) provided with at least one oblong housing (53) in which a drive spindle (54) is fixed.

3. The surgical instrument as claimed in claim 1, characterized in that the fixed jaw (3) consists of a cylindrical tube (23) comprising an internal bore (33) of a continuation (24) in the form of an elongate mounting plate having a U-shaped solid cross section and of a cylindrical shoulder (37) secured to a stop (38).

4. The surgical instrument as claimed in claim 1, characterized in that the transfer means consist of a shoulder (30) secured to the fixed jaw (3) and which enters the space (41) of the moving jaw (6) in the operating position.

5. The surgical instrument as claimed in claim 4, characterized in that the shoulder (30) comprises an elongate part (34) which is arranged inside the bore (33) and above the internal wall (35) of the cylindrical tube (23) of the fixed jaw (3) so as to delimit a slot (55) of curved profile in which the wall of the cylindrical tube (23) of the moving jaw (6) slides in the operating position.

6. The surgical instrument as claimed in claim 2, characterized in that the continuation (42) comprises, over its entire length, an internal passage (44) in the shape of an inverted U with an open base of a cross section increasing from the edge 45 as far as the shoulder 48.

7. The surgical instrument as claimed in claim 2, characterized in that the continuation (42) at its free end comprises inclined and opposed edges (45) which are beveled.

8. The surgical instrument as claimed in claim 2, characterized in that the passage (44) comprises, near the inclined edges (45), opposed ribs (46) separated by a slit (47) so as to constitute a slideway.

9. The surgical instrument as claimed in claim 2, characterized in that the ring (50) comprises oblong housings (53) which are made parallel to the longitudinal axis of the moving jaw (6), while drive spindles (54) are fixed in each housing are directed in a direction perpendicular to the longitudinal axis so as to collaborate with the moving handle (4) for driving the translation of the moving jaw (6).

10. The surgical instrument as claimed in claim 9, characterized in that the oblong housings (53) are uniformly distributed around the periphery of the ring (50).

11. The surgical instrument as claimed in claim 2, characterized in that the ring (50) comprises just one oblong housing (53) and just one drive spindle (54) while the tube (40) is free to rotate about the ring (50), allowing the moving jaw (6) to automatically position itself according to the angular indexing of the fixed jaw (3) relative to the fixed handle (2).

12. The surgical instrument as claimed in claim 3, characterized in that the continuation (24) comprises an upper edge (28) which has, at the shoulder (30) and inside the tube (23), lateral stops (31) delimiting, on the central axis of the fixed jaw (3), a groove (32) for the longitudinal guidance of the moving jaw (6) in the operating position.

13. The surgical instrument as claimed in claim 3, characterized in that the continuation (24) comprises, at its free end, a nose (25) which in its thickness has a hollowing (26) delimiting opposed and beveled edges (27).

14. The surgical instrument as claimed in claim 3, characterized in that the continuation (24) comprises an upper edge (28) provided, near the nose (25), with a T-shaped tenon (29) allowing longitudinal guidance of the moving jaw (6) in the operating position.

15. The surgical instrument as claimed in claim 5, characterized in that the wall (35) of the cylindrical tube (23) of the fixed jaw (3) is pierced with a hole (36) which communicates with the slot (55).

16. The surgical instrument as claimed in claim 3, characterized in that the tube (23) comprises, at the shoulder (37), a nut (39) provided in its internal part with a shoulder (49) which collaborates with the stop (38) to allow the fixed jaw (3) to be fixed indexed in a given angular position on the fixed handle (2).

17. The surgical instrument as claimed in claim 1, characterized in that the fixed handle (2) comprises fixing means that allow the fixed jaw (3) to be arranged in different angular positions about its longitudinal axis.

18. The surgical instrument as claimed in claim 17, characterized in that the fixing means consist of a cylindrical sleeve tube (8) comprising at one of its ends a threaded collar (15) delimiting a shoulder (16) in which cut-outs (17) are made, at least one of which collaborates, according to the determined position, with a stop (38) secured to the fixed jaw (3), while the threaded collar (15) collaborates with a tightening nut (39) for immobilizing the fixed jaw (3) on the fixed handle (2).

19. The surgical instrument as claimed in claim 18, characterized in that the cylindrical sleeve tube (8) comprises an internal bore (9) open at both ends and communicating with a slit (10) of conical profile formed in the thickness of the upper part of the fixed handle (2).

20. The surgical instrument as claimed in claim 19, characterized in that the slit (10) is designed to accommodate a mounting plate (20) of the moving handle (4) which opens into the internal bore (9) of the sleeve tube (8) so as to be able to collaborate with one of the drive spindles (54) that drive the moving jaw (6).

21. The surgical instrument as claimed in claim 20, characterized in that the mounting plate (20) of the moving handle (4) comprises an oblong slit (22) intended to accommodate one of the spindles (54) of the moving jaw in the operating position.
